(19) United States
(12) Patent Application Publication
(10) Pub. No.: US 2017/0032648 A1
(43) Pub. Date: Feb. 2, 2017

(54) PATIENT MONITORING SYSTEM

(71) Applicant: Eldersafe Technologies, Inc., Harvard, MA (US)

(72) Inventors: Michael McClain, Waterford, MI (US); Hans Mills, Livonia, MI (US); Leon Deligiannidis, Lexington, MA (US); Thomas Goulding, Harvard, MA (US)

(21) Appl. No.: 15/207,558

(22) Filed: Jul. 12, 2016

Related U.S. Application Data
(60) Provisional application No. 62/317,537, filed on Apr. 2, 2016.
(60) Provisional application No. 62/315,822, filed on Mar. 31, 2016.
(60) Provisional application No. 62/282,080, filed on Jul. 27, 2015.

(51) Int. Cl. G08B 21/04; A61B 5/00; A61B 5/11

(52) U.S. Cl. CPC G08B 21/043; A61B 5/1116; A61B 5/6894; G08B 21/0461

(57) ABSTRACT

A readily scalable, web-based system for monitoring the status of a plurality of patients by a plurality of caregivers includes a patient monitoring center that continuously aggregates patient data using patient monitoring devices, such as rounds tags and wheelchair sensors. The monitoring center compiles and stores a modifiable table that links each patient with a selection of caregivers and monitoring devices. The monitoring center additionally links certain caregivers with web-enabled compute devices, such as mobile telephones. Accordingly, upon detecting imminent risk of danger to a patient, such as detecting initiation of patient egress from a wheelchair, the monitoring center transmits a user-intuitive, real-time alert directly to the caregivers responsible for the well-being of the patient through their designated compute devices. In this manner, the risk of significant injury to the patient can be prevented. Additionally, historical data can be extracted and analyzed to ensure compliance with patient safety standards.

### FIELD OF THE INVENTION

The present invention relates generally to the healthcare industry and more particularly to systems for monitoring the well-being of a patient.

### BACKGROUND OF THE INVENTION

Routine patient monitoring has been found to be a critical component in the administration of effective health care services. In particular, among the growing elderly population, patient monitoring is not only required to ensure overall physical and mental well-being but, in increasing fashion, to limit the risk of elderly patients engaging in physical activity that could result in a fall. Presently, falls are the leading cause of injury and death among the elderly in Europe and America, with approximately 70 million elderly individuals experiencing a serious fall each year.

As can be appreciated, the routine monitoring of elderly patients is regarded as a highly effective fall mitigation, or fall prevention, strategy. Through frequent observation, the needs of a patient can be determined and satisfied which, in turn, limits the likelihood that the patient will engage in unassisted and potentially dangerous physical activity.

Currently, the use of patient monitoring as a fall mitigation strategy is commonly achieved using a variety of different techniques.

As a first example, patients are commonly monitored by conducting patient care rounds. As a part of the patient rounding process, a team of caregivers (e.g. doctors, nurses, family members and the like) evaluates the condition of a patient at defined intervals, logs relevant information relating to the perceived status of the patient, and dispenses any immediate treatment or assistance that is deemed necessary.

As a second example, patients are often passively monitored through the use of medical alert devices. A medical alert device is commonly constructed as a patient wearable device, often in the form of a bracelet or lanyard, which is designed to wirelessly transmit an alert condition when activated.

As a third example, patients are commonly monitored using automated monitoring devices, or sensors, which are designed to detect certain types of patient movement. Upon detecting a potentially dangerous condition (e.g. the patient leaving the chair), the sensor activates an alarm, which is often auditory or visual in nature, to notify caregivers that the well-being of the patient is at risk.

For instance, it is known in the art for automated monitoring devices to be installed onto wheelchairs, chairs and the like in order to detect patient activity that is indicative of impending egress. Examples of such devices are provided in U.S. Pat. No. 8,203,454 to Knight et al., and U.S. Pat. No. 6,204,767 to Sparks, the disclosures of which are incorporated herein by reference.

Data compiled through routine patient monitoring has been found to offer useful analytical value. For instance, patient monitoring data can be used for numerous purposes including, but not limited to, ensuring adherence to predefined institutional standards with respect to rounds schedules and alert response times, defending against accusations of patient neglect or improper care, and identifying potential changes in the behavior of patients and/or staff.

Although well-known and widely used in the art, patient monitoring systems that utilize one or more of the monitoring techniques set forth in detail above have been found to suffer from a few notable shortcomings.

As a first shortcoming, the various patient monitoring techniques set forth in detail above function in a largely disparate and independent fashion. As a result, patient monitoring data which is accumulated from patient rounds, medical alert devices, and automated monitoring sensors is not typically aggregated into a single central repository. As a result, there currently exists no effective means to thoroughly evaluate the complete scope of care afforded to a patient.

As a second shortcoming, most conventional monitoring techniques are largely passive in nature and thereby fail to anticipate and warn of potentially dangerous conditions before harm is rendered to the patient. Stated simply, traditional patient monitoring systems focus primarily on detecting falls rather than preventing falls. As a result, conventional systems fail to remedy the increase in fall rates among the elderly.

As a third shortcoming, traditional monitoring systems offer limited scalability and adjustability to meet the needs of the patient. Most notably, the detection of an alert condition most often triggers a single alarm or notification at a fixed location, such as a nurse station or a monitor in close proximity to the patient. Because patients are commonly under the supervision of a team of healthcare personnel, many of whom are routinely moving across different sites, greater system efficiency and, in particular, alert response times would be achieved through notification directly to a user-modifiable selection of the personnel.

As a fourth shortcoming, traditional monitoring systems often fail to operate in real time. Rather, patient rounds data is often compiled and uploaded into a computer system at designated intervals (e.g. at the end of a shift). Further, patient rounds are commonly rendered in accordance with a predefined schedule that does not accommodate for changes in the status of certain patients. For obvious reasons, a lack of real-time alerts and notifications creates a potential harmful environment for a patient. By contrast, with proper and timely forewarning, a potentially hazardous condition could be immediately recognized and remedied before any patient harm is endured.

As a fifth shortcoming, traditional monitoring systems are often ineffective in rendering suitable patient care. In particular, it has been found that practitioners not only routinely encounter false alarms but also often incur difficulty in determining which patient is experiencing an alert condition due to the limited information provided from conventional alarms.

### SUMMARY OF THE INVENTION

It is an object of the present invention to provide a new and improved system for monitoring patients.

It is another object of the present invention to provide a system as described above that aggregates all patient monitoring data into a central database.

It is yet another object of the present invention to provide a system as described above that readily and accurately anticipates potentially dangerous patient conditions.

It is still another object of the present invention to provide a system as described above that directly notifies a user-modifiable selection of caregivers of potentially dangerous patient conditions.

It is yet still another object of the present invention to provide a system as described above that compiles patient status data and issues pertinent alerts and notifications in real time.

It is another object of the present invention to provide a system as described above that is highly intuitive, efficient and self-administering in operation, readily scalable in size, and inexpensive to implement.

Accordingly, as one feature of the present invention, there is provided a system for monitoring of the status of a plurality of patients by a plurality of caregivers, a selection of the plurality of caregivers being assigned to each of the plurality of patients, the system comprising (a) a central controller for aggregating and evaluating data relating to the status of each of the plurality of patients, (b) a plurality of patient monitoring devices, each of the plurality of patient monitoring devices being in close proximity to one of the plurality of patients, the plurality of patient monitoring devices being in electronic communication with the central controller, and (c) a plurality of compute devices, at least one of the plurality of compute devices being in close proximity to a corresponding caregiver, the plurality of compute devices being in electronic communication with the central controller, (d) wherein the central controller is adapted to receive data relating to the status of each of the plurality of patients from at least one of the plurality of patient monitoring devices and the plurality of compute devices, the central controller selectively transmitting alerts and notifications relating to the status of a patient to the corresponding selection of assigned caregivers for the patient via one or more designated compute devices.

Various other features and advantages will appear from the description to follow. In the description, reference is made to the accompanying drawings which form a part thereof, and in which is shown by way of illustration, an embodiment for practicing the invention. The embodiment will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings wherein like reference numerals represent like parts:

FIG. 1 is a simplified block diagram of a patient monitoring system constructed according to the teachings of the present invention;

FIGS. 2(a) and 2(b) are front perspective and exploded, top perspective views, respectively, of one of the patient monitoring devices shown in FIG. 1, the patient monitoring device being constructed according to the teachings of the present invention;

FIG. 3 is a simplified flow chart which represents an overview of the functional operation of the patient monitoring device shown in FIG. 2(a);

FIGS. 4(a) and 4(b) are front perspective and rear perspective views, respectively, of the patient monitoring device shown in FIG. 2(a), the patient monitoring device being shown mounted on a wheelchair;

FIG. 5 is a simplified flow chart which represents an overview of a novel method for minimizing the risk of patient falls, the method being described herein according to the teachings of the present invention and implemented using the patient monitoring system shown in FIG. 1; and

FIGS. 6(a)-(i) are a series of sample screen displays which are useful in understanding how certain features of the method shown in FIG. 5 can be accessed using a mobile device.

### DETAILED DESCRIPTION OF THE INVENTION

### Patient Monitoring System 11

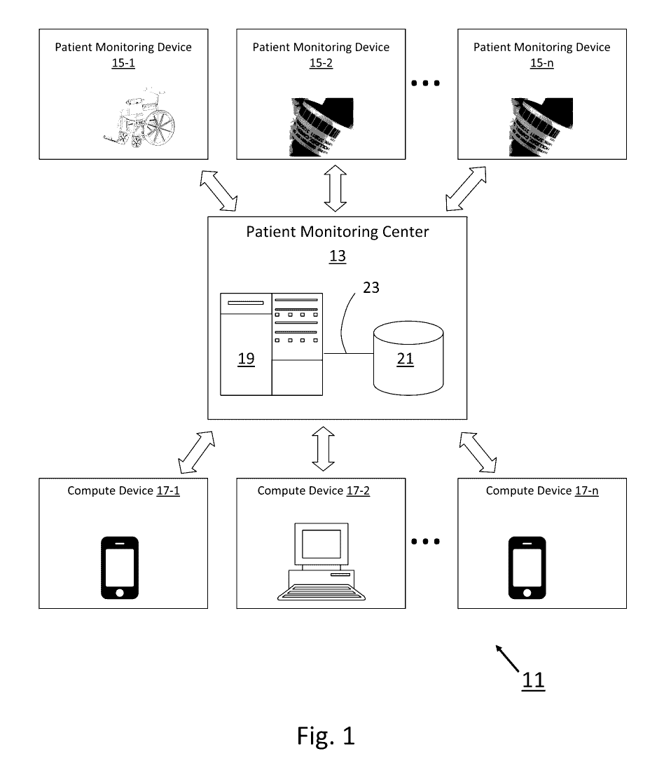

Referring now to FIG. 1, there is a shown a simplified block diagram of a patient monitoring system designed according to the teachings of the present invention, the patient monitoring system being identified generally by reference numeral 11. As will be described further below, patient monitoring system 11 is a comprehensive, highly efficient, and self-administrating computer platform that relies principally on (i) routine visual patient observation, (ii) automated identification of imminent patient egress from a wheelchair or similar structure, and (iii) rapid and direct caregiver notifications to maximize patient safety.

As can be seen, system 11 is designed with a scalable architecture that comprises a patient monitoring center 13 which serves as the central hub for facilitating the exchange of data between a plurality of patients and a plurality of caregivers to ensure that patient care standards are maintained. Specifically, patient monitoring center 13 is electronically linked with a plurality of patients via patient monitoring devices 15-1 thru 15-n and a plurality caregivers via compute devices 17-1 thru 17-n.

Patient monitoring center 13 comprises a central controller 19 and a data storage device, or database, 21 in electronic communication with one another via network path 23. As can be appreciated, central controller 19 serves as the functional hub of system 11 that aggregates patient data for storage in database 21 and, in turn, automatically issues any alerts and/or notifications to a user-modifiable selection of caregivers via devices 17.

Central controller 19 is represented herein as a cloud server that regulates the exchange of data between the principal participants of system 11. Accordingly, as a feature of the present invention, system 11 operates as a cloud-based solution, which provides for greater ease of scalability and facilitates software updates. However, it is be understood that system 11 could be alternatively configured as a local, on-premises solution (e.g. for enhanced security of patient data) without departing from the spirit of the present invention.

As noted above, monitoring devices 15 and compute devices 17 are directly linked with central controller 19 through any appropriate communication path that is suitable for the electronic transfer of data, such as the internet. However, it is to be understood that a local server, or other similar intermediary device, may be provided at a patient facility to facilitate communication between controller 19 and devices 15 and 17, thereby allowing for seamless integration with preexisting computer networks.

As a feature of the present invention, patient monitoring devices 15 can take a variety of different forms. For instance, patient monitoring device 15-1 is represented herein as a novel wheelchair sensor that is specifically designed to promptly and accurately identify movement indicative of impending chair egress, the details of wheelchair sensor 15-1 to be described further in detail below.

Additionally, each of patient monitoring devices 15-2 and 15-n is represented as a patient, or rounds, tag which includes a unique patient identification code that can be automatically retrieved using automatic identification and data capture (AIDC) technologies (e.g. using barcode, QR code, or RFID technologies). Using an appropriate reader (e.g. a properly equipped mobile device 17), the patient identification code can be automatically captured and transmitted to patient monitoring center 13 with a corresponding time/date stamp. Using such information, interested parties (e.g. medical administrators and family members) can track the frequency that a patient is visited for a particular period of time.

Compute devices 17 represent any machine capable of interfacing with cloud-based central controller 19. For example, each compute device 17 may be in the form of a web-enabled mobile device that interfaces with central controller 19 through a designated software application (e.g. compute device 17-1), a fixed, desktop computer that interfaces with central controller 19 via the internet (e.g. compute device 17-2), a portable tablet computer (not shown), or a web-enabled, high-definition television monitor (not shown).

The ability for standard mobile devices to be readily integrated into system 11 by downloading the designated software application significantly enhances the overall scalability of the platform. Further, the portable nature and ubiquitous use of mobile devices ensures that any alerts and/or notifications transmitted to a particular caregiver are immediately received, which is highly desirable.

### Patient Monitoring Device 15-1

As referenced above, patient monitoring device 15-1 is in the form of a novel wheelchair sensor that is specifically designed to promptly and accurately identify movement indicative of impending chair egress. In response thereto, device 15-1 automatically transmits a signal to central controller 19 that is indicative of the stage of patient egress. If the movement exceeds a predefined threshold, central controller 19 can instantly, automatically and directly notify certain caregivers of the change in patient status via his/her compute device 17.

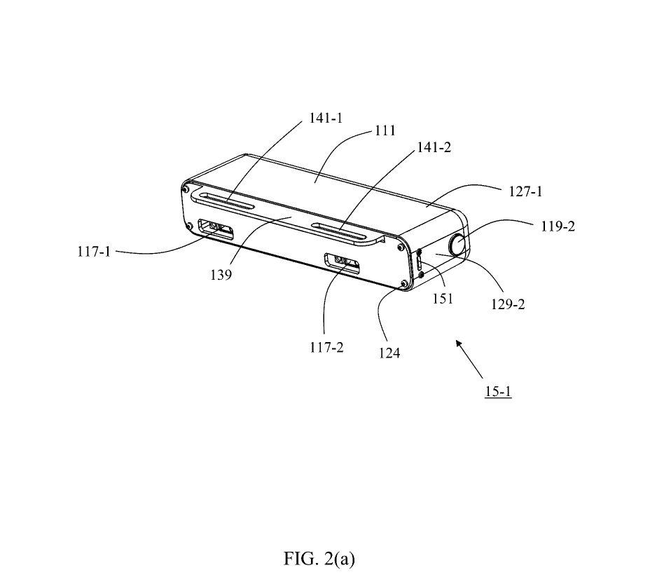

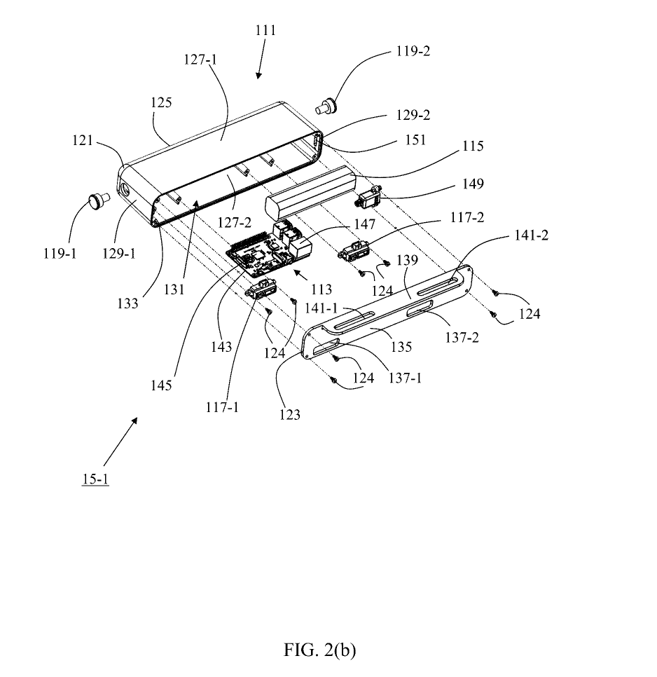

Referring now to FIGS. 2(a) and 2(b), patient monitoring device 15-1 is constructed as a unitary member that is compact in its design. Accordingly, as will be explained further below, device 15-1 is particularly well suited to be removably secured to a wheelchair in an unobtrusive manner.

Device 15-1 comprises an outer housing 111, electronics 113 for controlling the principal operations of device 15-1, a battery 115 for supplying the necessary power to electronics 113, a pair of motion sensors 117-1 and 117-2 for detecting patient movement, and a pair of control buttons 119-1 and 119-2 for manually regulating certain operations of device 15.

As can be seen, outer housing 111 is in the form of a compact, generally rectangular case that is preferably constructed of a rigid and durable material, such as plastic. Housing 111 includes a base 121 and a cover 123 that are releasably secured together by screws 124.

Base 121 comprises a bottom wall 125, a pair of sidewalls 127-1 and 127-2, and a pair of end walls 129-1 and 129-2 that together define an interior cavity 131 dimensioned to receive various components of device 15-1. Access to interior cavity 131 is provided through an open top 133.

Cover 123 comprises a generally flat plate 135 that is dimensioned to substantially enclose open top 133 of base 121. A pair of spaced apart, transverse slots 137-1 and 137-2 is formed in plate 135 and provides openings through which sensors 117-1 and 117-2, respectively, can monitor patient movement, as will be explained further below.

Cover 123 additionally includes a planar flange 139 that extends orthogonally out from the outer surface of plate 135 along its top edge. Flange 139 is shaped to define a pair of spaced apart, transverse slots 141-1 and 141-2 that facilitate securing housing 111 onto a wheelchair, as will be explained further below.

As noted above, electronics 113 is responsible for controlling the principal operations of device 15-1. For ease of assembly, the various electrical components for electronics 113 are preferably mounted on a common circuit board 143, which in turn is fixedly mounted within cavity 131 in base 121. Among other things, electronics 113 includes a programmable controller 145 for regulating control of device 15 and a transmitter 147 for wirelessly sending communication signals (e.g. relating to patient movement) to central controller 19 using conventional communications protocols, such as Transmission Control Protocol/Internet Protocol (TCP/IP).

It should be noted that transmitter 147 preferably sends signals at defined intervals (e.g. at 60 second intervals). Consequently, if a signal is not received by central controller 19 in a timely fashion, the patient monitoring device 15-1 is considered by center 13 to be non-operational. In turn, appropriate notifications can be sent to devices 17 to ensure that patient monitoring is not expected from a non-operational device 15.

It should also be noted that each signal sent by transmitter 147 can be tracked using conventional Global Positioning System (GPS) technologies. Accordingly, it is to be understood that monitoring center 13 is able to continuously track the geolocation of each wheelchair sensor 15-1 and thereby determine if a patient leaves a designated area (e.g. a healthcare facility).

Sensors 117-1 and 117-2 are fixedly mounted within cavity 131 of base 121 by screws 124 so as to align directly with slots 137-1 and 137-2, respectively. Each sensor 117 is preferably an infrared distance sensor that is in electrical communication with electronics 113. Although not shown herein, filters may be provided along the communication path between each sensor 117 and controller 145 in order to stabilize the detection signal.

Any motion detected by sensors 117 is received and analyzed by programmable controller 145 in order to determine notable patient movement. As will be explained below, controller 145 is provided with a program that applies changes in patient position against a proprietary algorithm in order to limit the transmission of an alert condition to only such movement that is indicative of a patient initiating the process of standing. In other words, controller 145 is programmed such that routine, minimal movement within the chair (e.g. ordinary patient readjustment or slouching) does not trigger an alert condition.

Specifically, the dual sensor construction of device 15-1 preferably enables patient movement to be evaluated based on two principal components: (1) posterior forward (PF) movement—sliding, or scooting, forward to the edge of the seat, and (2) lean forward (LF) movement—forward leaning posture about the waist. As will be explained further below, sensor data is relied upon by the fall mitigation algorithm to accurately detect initiation of chair egress, rather than ordinary slouching or fidgeting within a chair that does not indicate initiation of the standing process.

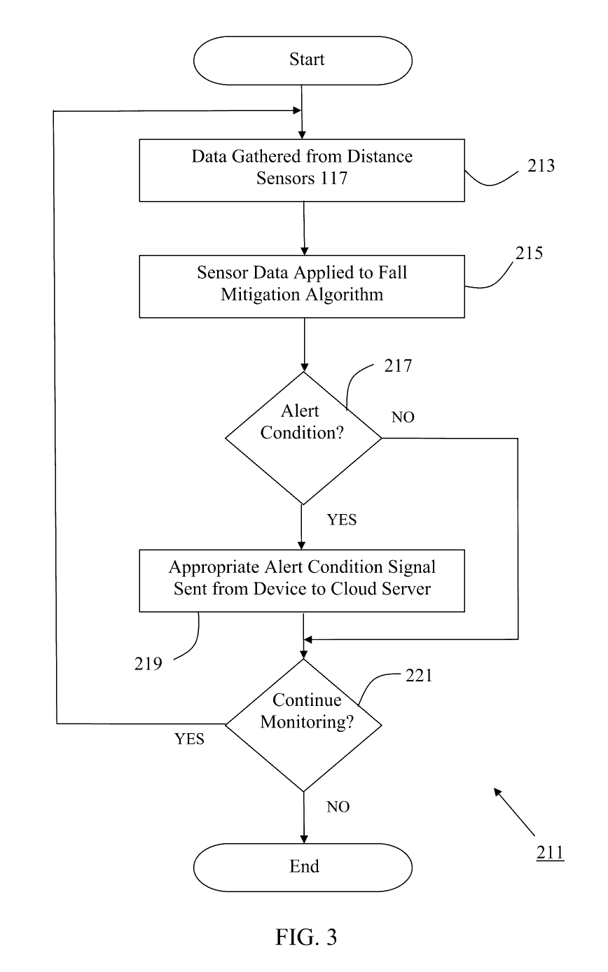

Referring now to FIG. 3, there is shown a simplified flow chart which represents the process by which device 15-1 determines the presence of an alert condition, the process being identified generally by reference numeral 211. In the first step of process 211, distance data is continuously gathered by sensors 117 and sent to controller 145, the data gathering step being identified by reference numeral 213.

Upon the detection of movement, sensors 117 measure the relative location of the patient within the wheelchair and apply the sensor data against the fall mitigation algorithm in step 215. As a feature of the present invention, the fall mitigation algorithm detects different levels, or gradients, of patient movement based on whether the sensor data reaches certain predefined thresholds. Most notably, the algorithm preferably determines that the physical location of the patient within the chair is indicative of (1) a non-alert condition, color coded as green, when sensors 117 detect that the patient is safely positioned in the rear of the chair, (2) a low alert condition, color coded as yellow, when sensors 117 detect that the patient has slid forward to mid-chair, thereby indicating possibility of egress, (3) a high alert condition, color coded as red, when sensors 117 detect that the patient has slid forward to the front edge of the chair, thereby indicating initiation of egress, and (4) a fall condition, color coded as blue, when sensors 117 detect no patient in the chair, thereby indicating chair egress and immediate risk of fall.

Controller 145 then determines whether a positive alert condition, either low, high or fall, is present in step 217. It should be noted that accumulated information about the patient, such as environmental factors (e.g. time of day, elapsed time since last round, etc.), specific medical conditions, and historical data (e.g. patient movement statistics), may be considered by controller 145 when determining the presence of an alert condition. As can be appreciated, consideration of such factors serves to improve the overall accuracy in determining an alert condition.

If an alert condition is detected, a signal corresponding to the detected level of movement is automatically sent in real time from device 15-1 to central controller 19 in step 219. Thereafter, or if no alert condition was detected back in step 217, process 211 determines whether the patient requires further monitoring in step 221. If further monitoring is required, process 211 returns back to step 213 and repeats as such throughout the monitoring period. Otherwise, process 211 terminates.

Referring back to FIGS. 2(a) and 2(b), battery 115 is disposed within cavity 131 of base 121 and supplies electronics 113 with power. Preferably, battery 115 is of the low power (e.g. 5 volt) and rechargeable variety. Accordingly, a power jack 149 is disposed within cavity 131 of base 121 and is electrically coupled to battery 115. External access to power jack 149 for recharging purposes is achieved through a slot 151 provided in end wall 129-2 that is in alignment with power jack 149. Although not shown herein, an indicator (e.g. a plurality of light emitting diodes) may be incorporated into device 15-1 to display the current power level of battery 115.

Externally accessible control buttons 119-1 and 119-2 fittingly penetrate through corresponding openings in end walls 129-1 and 129-2, respectively, and are electromechanically connected to electronics 113. In use, the manual depression of each control button 119 creates a corresponding signal that is received by electronics 113.

In this manner, control buttons 119 provide means for manually regulating certain operations for device 15. Notably, control button 119-1 is designed as a call button which the patient can activate in order to call the attention of designated caregivers (e.g. if the patient requires immediate assistance). Power button 119-2 is designed as a power button which can be used to temporarily deactivate and subsequently reactivate patient monitoring device 15-1. Although not shown herein, an indicator, such as a single light emitting diode, may be incorporated into device 15-1 to denote when device 15-1 is in active operational state.

It should be also noted that, in addition to power button 119-2, activation of monitoring device 15-1 could be achieved using a compute device 17 that is installed with the required software application. Using such an application, activation of device 15-1 could be accomplished, for example, by touching a user-intuitive button in the software application and/or using Near Field Communication (NFC) technology. In this manner, a caregiver could temporarily disable device 15-1 when properly assisted egress from the chair occurs. Further, it is envisioned that a signal notifying activation of device 15-1 by a compute device 17 could be transmitted to patient monitoring center 13 with a corresponding time/date stamp in order to commence compilation of historical data relating to patient visits (i.e. to ensure compliance with rounds standards).

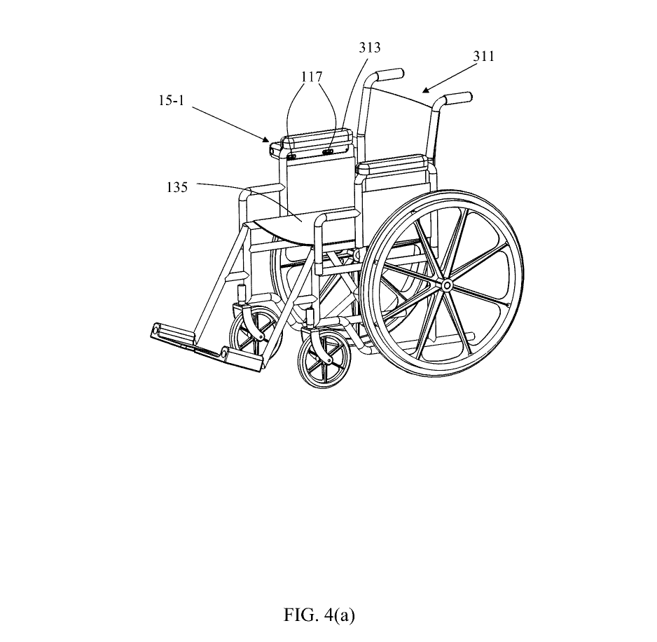

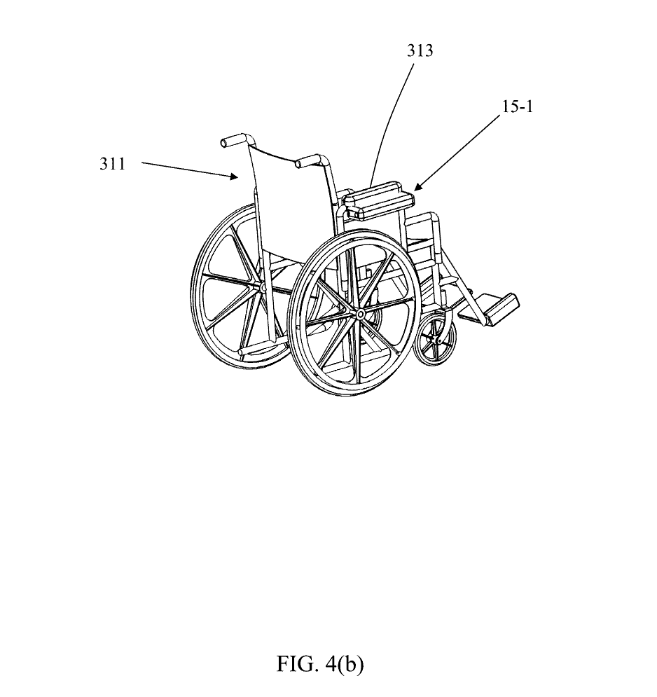

As noted briefly above, patient monitoring device 15-1 is specifically designed to be removably secured to a wheelchair in an unobtrusive manner so as to not impart any potential discomfort to the patient or prevent mounting of an oxygen tank or other similar item thereon. Specifically, in FIGS. 4(a) and 4(b), patient monitoring device 15-1 is shown mounted onto a conventional wheelchair 311. As can be seen, device 15-1 is designed to be secured to the underside of the right side armrest 313 for wheelchair 311.

Preferably, wheelchair 311 is standard medical wheelchair, such as the VERANDA line of wheelchairs manufactured by Invacare Corporation of Elyria, Ohio. Accordingly, as part of the construction of wheelchair 311, a pair of screws (not shown) is typically driven into the underside of armrest 313. After temporarily removing these preexisting screws, device 15-1 is disposed such that flange 139 of outer housing 111 lies flush against the underside of armrest 213, with sensors 117 directed inward towards a patient sitting on seat 315 of wheelchair 311, as seen in FIG. 4(a). The screws are then driven through slots 141 in flange 139 and back into threaded engagement with the preexisting bores formed in the underside of armrest 313, thereby fixedly securing device 15-1 to wheelchair 311.

In this manner, it is to be understood that device 15-1 need not be permanently affixed to a single wheelchair. Rather, device 15-1 could be removed from one wheelchair and reused on other wheelchairs, as needed. As such, preexisting wheelchairs can be retrofit with device 15-1, as needed, in order to provide patient monitoring capabilities.

Moreover, it should be noted that device 15-1 is not limited for use with a wheelchair. Rather, it is to be understood that device 15-1 could be modified in its construction to allow for securement to alternative items typically found in a healthcare facility without departing from the spirit of the present invention.

As an example, it is to be understood that device 15-1 could be modified in construction to allow for securement and use with a medical walker. In this manner, caregivers can be immediately notified when sensors determine that a patient has moved away from the walker and, as such, is at a heightened risk of falling.

As another example, it is to be understood that device 15-1 could be modified in construction to allow for securement and use with a bed. In this manner, caregivers can be immediately notified when sensors determine that a patient has moved to the edge of the bed and may attempt to stand without assistance.

As yet another example, it is to be understood that device 15-1 may be simplified, or stripped down, in its construction in order to function solely as a call device for manually notifying caregivers of an immediate request by a patient for assistance. In other words, sensors 117 and other related components that do not relate to the call feature of device 15-1 would preferably be removed therefrom to simply construction and minimize manufacturing costs.

### Patient Monitoring Method 411

As referenced above, system 11 is a comprehensive, all-in-one, cloud-based platform for ensuring the well-being of patients. As will be described further in detail below, system 11 utilizes a novel patient monitoring method 411 that effectively minimizes the risk of patient falls by, inter alia, (i) tracking compliance with scheduled patient rounds and (ii) identifying patient movement that indicates imminent egress from a chair. In turn, system 11 automatically issues alerts and notifications directly to a user-modifiable selection of caregivers via devices 17 as part of an effective fall mitigation solution.

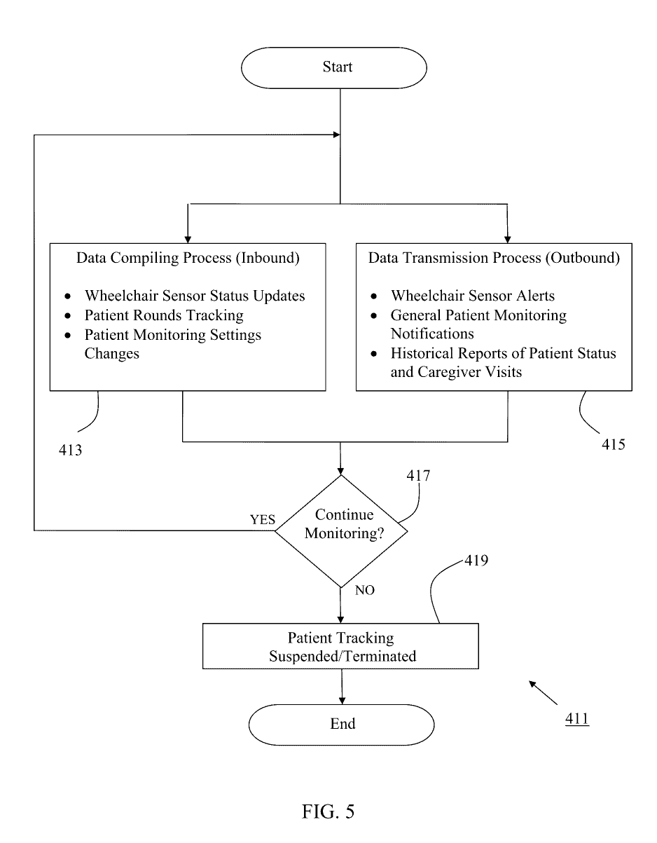

As seen in FIG. 5, method 411 relies upon two principal processes, or steps, that operate continuously and concurrently throughout the duration of the patient monitoring cycle. Specifically, method 411 includes (i) a data compiling, or inbound, step 413 in which patient monitoring information is aggregated by central controller 19 and (ii) a data transmission, or outbound, step 415 in which patient monitoring information is automatically sent, in real time, from central controller 19 to a user-modifiable selection of caregivers via devices 17. In this manner, method 411 is able to continuously compile critical patient data obtained through various manual and automated means and, in turn, send appropriate alerts, notifications, and reports to selected individuals, when necessary.

Data compiling step 413 includes three principal categories of data received by central controller 19, namely, (i) color-coded status changes detected by wheelchair sensors 15-1, (ii) rounds tracking data, which includes the exact time and date that a patient calls a caregiver and the exact time and date that a caregiver conducts a rounds visit, and (iii) general modification of patient monitoring settings including, but not limited to, adding/removing patients, adding/removing caregivers assigned to each patient, pausing/resuming patient monitoring sessions, adjusting round schedules, modifying alarm types, and selecting which caregivers are to receive certain alerts and notifications for each patient. By means of step 413, patient monitoring center 13 is able to accumulate and aggregate all relevant patient-related information that is obtained from a wide range of previously disparate sources.

Data transmission step 415 includes three principal categories of data sent from central controller 19 to selected compute devices 17, namely, (i) alerts of notable status changes detected by wheelchair sensors 15-1 (e.g. indicating that the patient has left the wheelchair and is in immediate danger of a fall), (ii) general monitoring notifications relating to, inter alia, patient calls, monitoring rounds reminders (e.g. monitoring session paused, rounds completed, time of last round visit for a particular patient, etc.) and patient monitoring setting changes (e.g. new patients, lists of active devices 15, adjustment of scheduled round times, etc.), and (iii) historical reports of patient status and caregiver visits for review by supervisors and administrators to ensure that fall mitigation protocols are being followed.

As noted above, steps 413 and 415 run continuously and concurrently throughout the duration of patient monitoring process 411. At any time, a decision can be made whether to suspend or terminate monitoring process 411 (e.g. if the patient requires use of the restroom under assisted supervision or is leaving the care of the facility), this decision step being represented by reference numeral 417. If the monitoring process is to be suspended or terminated, the patient is removed from tracking by system 11 (e.g. using a handheld compute device 17) in step 419 and method 411 terminates. Otherwise, if continued patient monitoring is required, method returns back to steps 413 and 415.

### Illustrative Example of Patient Monitoring Method 411

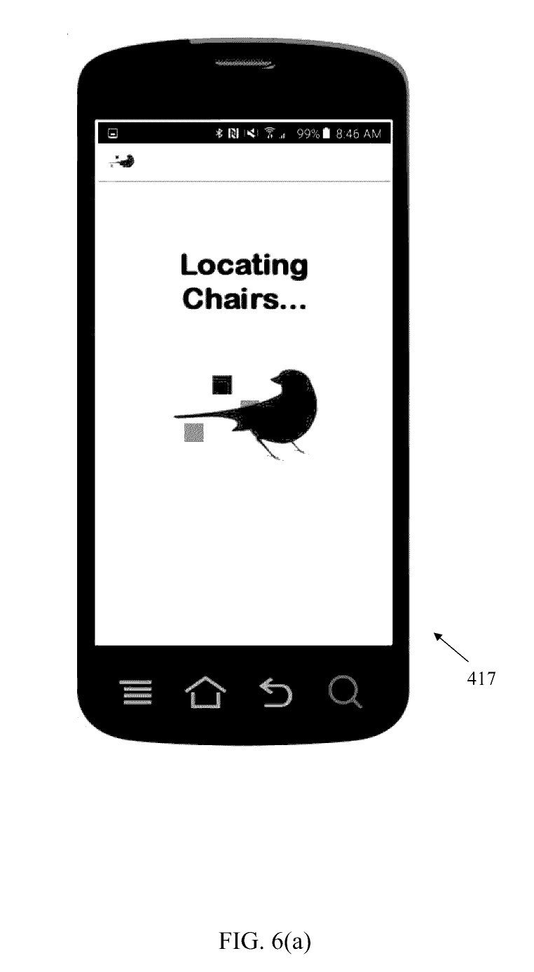

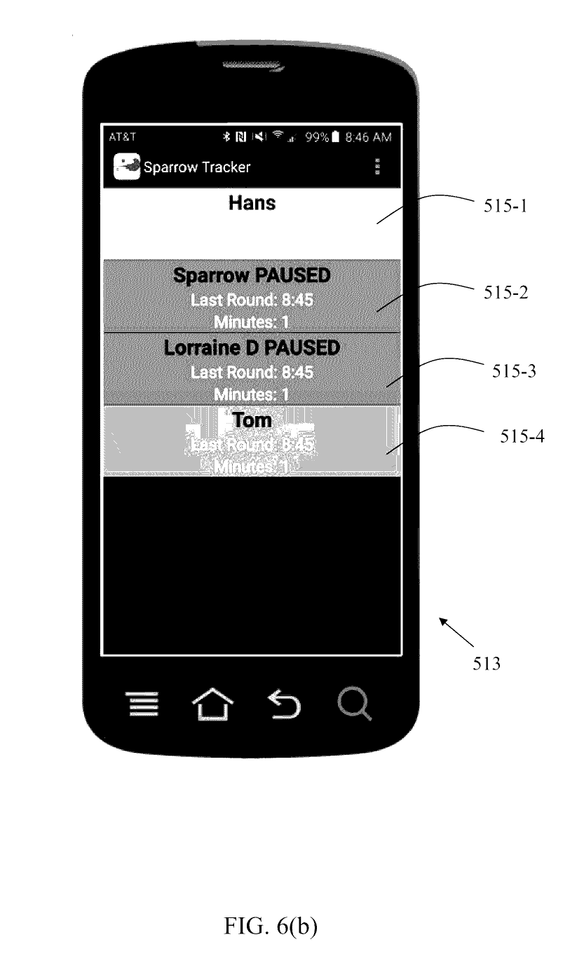

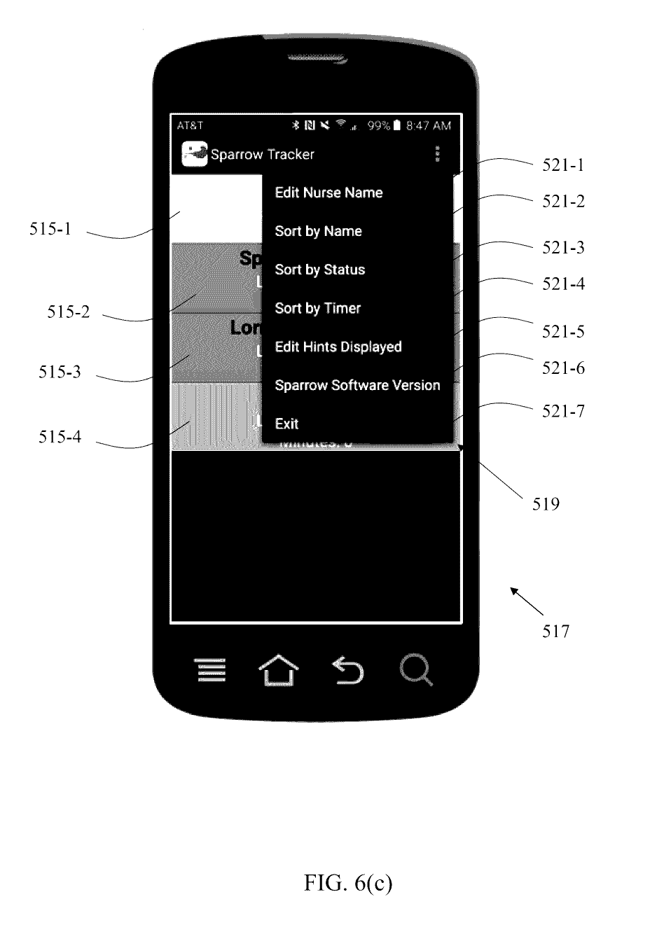

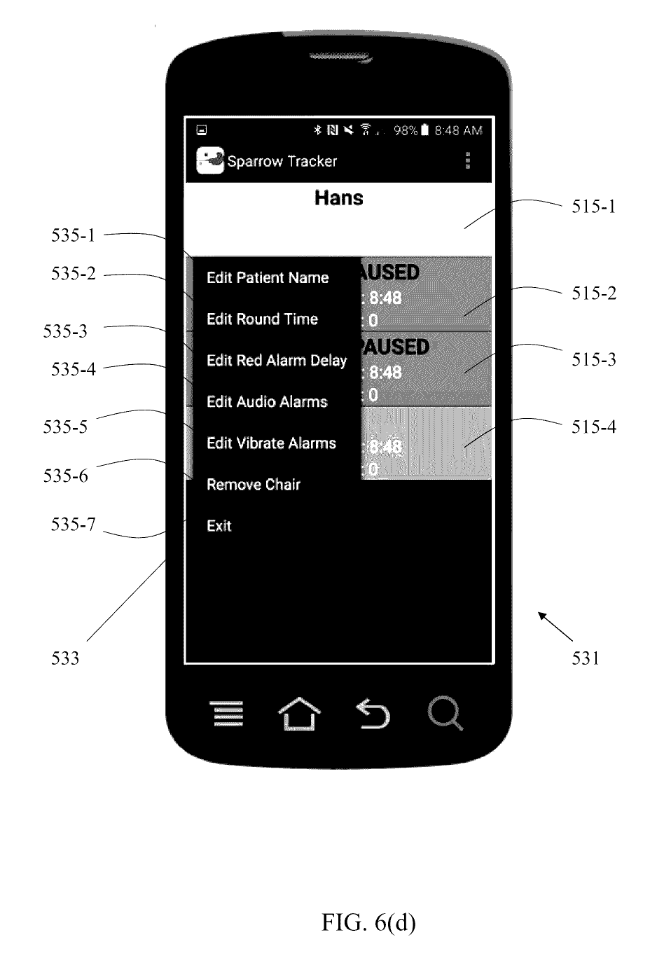

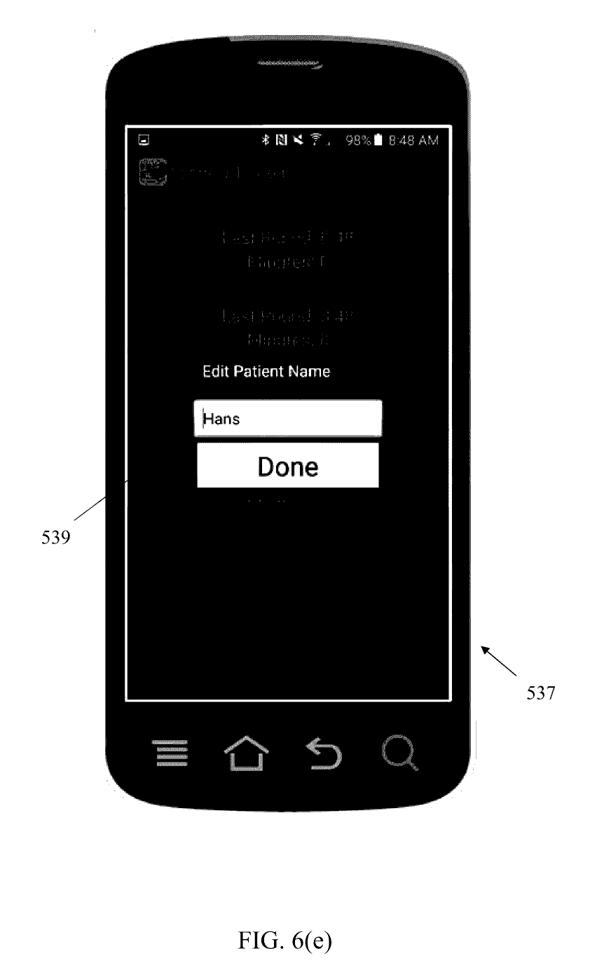

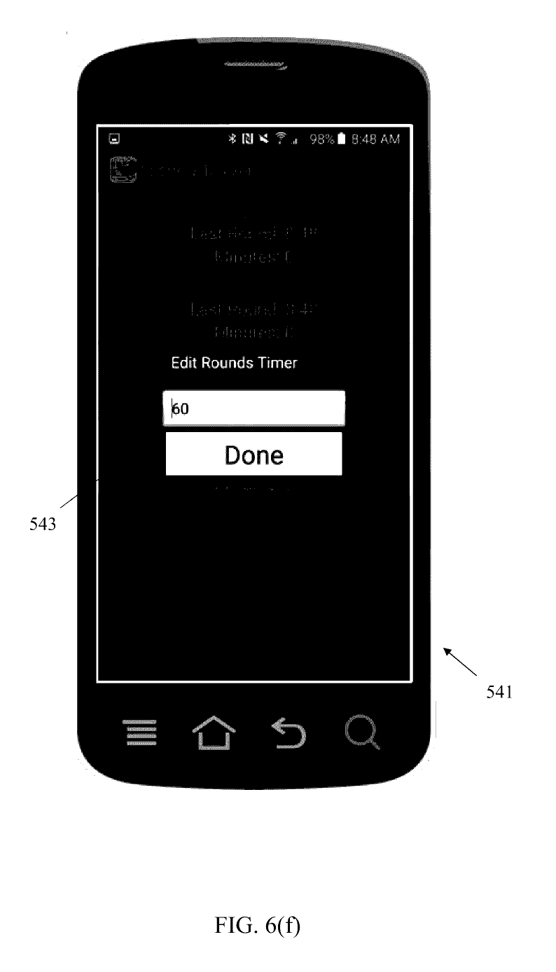

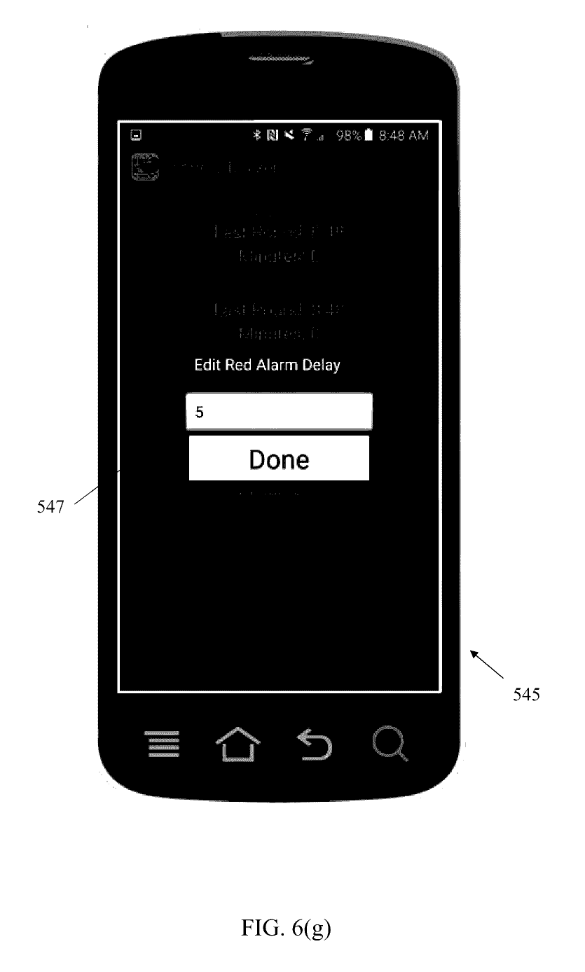

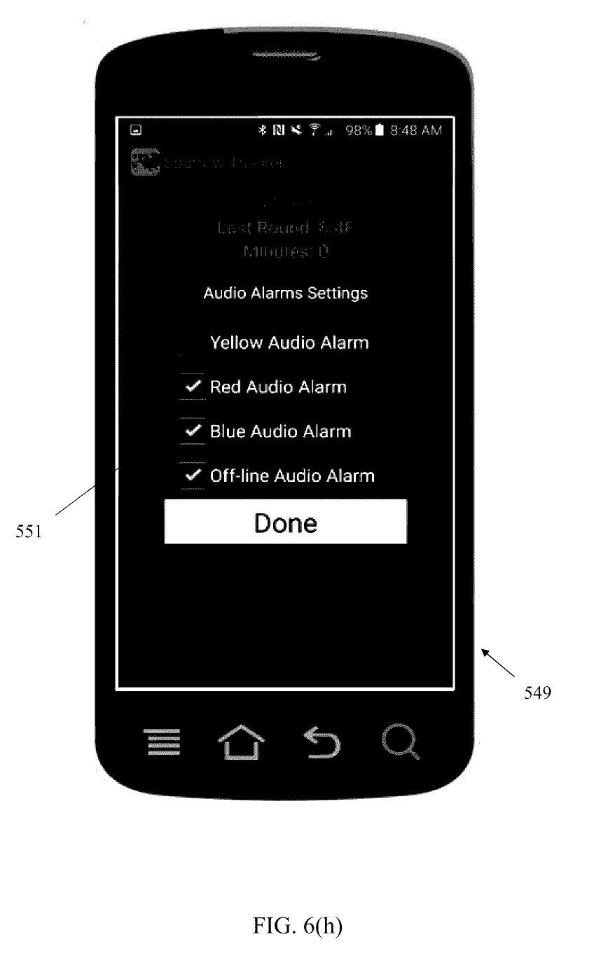

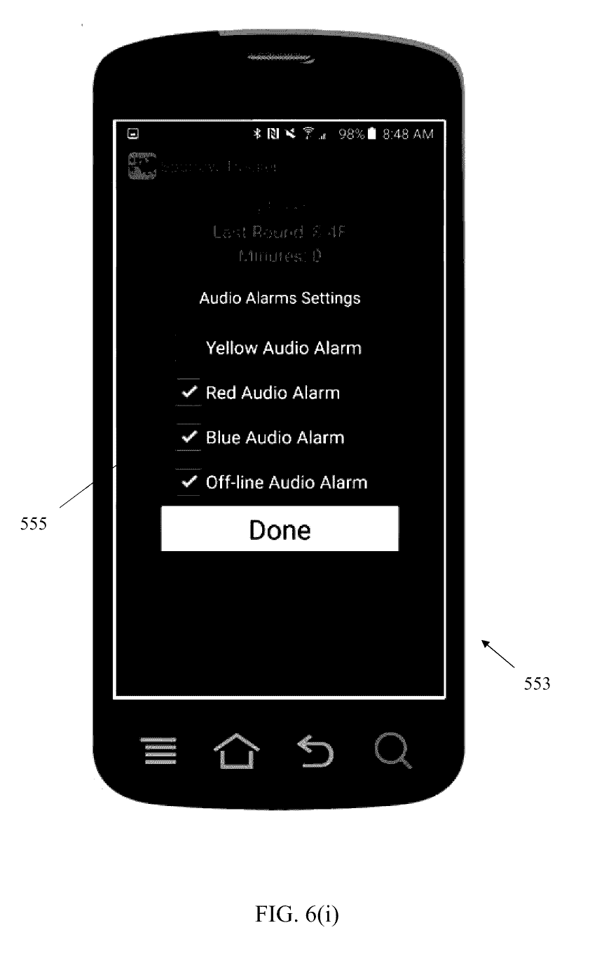

For ease in understanding the details of patient monitoring method 411, an illustrative example is provided herein. Specifically, referring now to FIGS. 6(a)-(i), there are shown a series of sample screen displays on a mobile device (e.g. device 17-1) that are helpful in understanding certain notable features of patient monitoring method 411.

Specifically, as referenced above, an individual caregiver responsible for monitoring the well-being of a patient can be seamlessly integrated into system 11 using a standard, web-enabled mobile device 17. The caregiver is simply required to connect with central controller 19, either by accessing a designated webpage or downloading a specified software application, and create a caregiver account in order to verify the propriety of incorporation within system 11 (i.e. to prevent unauthorized individuals from accessing information from database 21).

In a similar fashion, additional monitoring devices 15 can be seamlessly integrated into system 11. For instance, wheelchair sensor 15-1 can be integrated into system 11 by simply depressing control button 119-2 and uploading any login information required to access the local wireless network at the healthcare facility (e.g. by connecting a flash drive containing the login information to a designated universal serial bus (USB) communication port (not shown) in device 15-1).

Thereafter, using the designated software application, a mobile device 17 is able to complete the setup process for integrating patient monitoring device 15-1 into system 11. Specifically, mobile device 17 retrieves the unique identification code directly from the wheelchair sensor 15-1 (e.g. using Near Field Communication or barcode scanning technologies). In turn, device 17 is used to assign a patient name or other similar identifier (e.g. a room number), the caregiver identification code, and the facility name (if system monitoring occurs across multiple facilities) with the unique ID code for wheelchair sensor 15-1. The linked information is then transmitted from mobile device 17 to central controller 19 and is stored as such within a patient monitoring table in database 21. In this capacity, all relevant information relating to a patient monitoring device 15 (e.g. the device identifier, patient name, any caregiver identifiers and facility name) can be stored in central database 21 without the need of a complex data entry process, thereby minimizing administrative overhead.

With the all devices 15 and 17 properly linked and setup with system 11, a caregiver can routinely connect with patient monitoring center 13 in order to (i) transmit patient data to central controller 19 (e.g. rounds data, patient status) and, at the same time, (ii) receive real-time alerts and notifications. In this manner, the well-being of multiple patients can be effectively tracked using one or more standard mobile devices.

After login with central controller 19, each compute device 17 is initially directed to a start screen of the type represented in FIG. 6(a), which is identified generally by reference number 511. As can be seen, upon initial connection of mobile device 17 with patient monitoring center 13, central controller 19 extracts from the appropriate table in database 21 which patients, as well as the identification codes of monitoring devices 15 linked with such patients, are presently assigned to the caregiver. Thereafter, controller 19 determines the real-time status of each of the group of patients under the responsibility of the caregiver and transmits the status information to his/her mobile device 17.

Referring now to FIG. 6(b), there is shown a sample screen display that displays the status of all patients assigned to the caregiver, the patient status screen display being identified generally by reference number 513. As can be seen, display 513 includes a list of real-time, patient status blocks 515-1 thru 515-4, the order of which can be modified by the caregiver, as will be explained further below.

Each patient status block 515 includes easily discernible, user-intuitive information regarding the current state of the patient. Specifically, each status block 515 preferably includes (i) the name of the patient or other simple recognizable identifier (e.g. Hans), (ii) the time of the last patient visit (e.g. 8:45), and (iii) a real-time counter that displays the elapsed time since the last patient visit (e.g. Minutes: 1). Using this information, the status of all patients under the responsibility of the caregiver can be easily monitored.

Additionally, each patient status block 515 is preferably color coded to provide a simple and intuitive means to quickly assess the status of each patient. For instance, as referenced in detail above, a non-alert condition is preferably color coded as green, and notifies the caregiver that no immediate attention is required for the patient. However, low, high and fall alert conditions, which are preferably color coded as yellow, red and blue, respectively, notifies the caregiver, at different levels of urgency, that the patient requires attention. Lastly, for any patient monitoring device 15 that is temporarily disabled, either intentionally or unintentionally, the corresponding patient status block 515 is preferably color coded as gray.

It should be noted that the information provided on screen display 513 can be modified to the particular preferences of the caregiver. Specifically, referring now to FIG. 6(c), there is shown a sample patient status screen display which is useful in understanding how a caregiver can modify the patient status display settings, the sample screen display being represented by reference numeral 517.

As can be seen, a dropdown box 519 has been activated which includes (i) an edit nurse name button 521-1 for changing the nurse name assigned to the mobile device 17, (ii) a sort by name button 521-2 for sorting patient status blocks 515 alphabetically based on patient names, (iii) a sort by status button 521-3 for sorting patient status blocks 515 based on current color levels, (iv) a sort by timer button 521-4 for sorting patient status blocks 515 numerically based on running rounds counters, (v) an edit hints displayed button 521-5 for regulating whether to activate pop-up windows that provide assistance on how to utilize the application, (vi) a software version button 521-6 for displaying information relating to the current software version in use, and (vii) an exit button 521-7 for closing dropdown box 519.

With the setup of wheelchair sensor 15-1 and caregiver mobile device 17 completed, patient data can be transmitted to central controller 19 and logged in database 21 in three principal ways.

First, as referenced above, wheelchair sensor 15-1 is adapted to continuously monitor patient movement. If the fall mitigation algorithm detects a notable change in patient posture, position and movement, wheelchair sensor 15-1 automatically transmits the status change to central controller 19, which is then logged into database 21.

Second, as referenced above, patient rounds can be logged with patient monitoring center 13 using caregiver mobile devices 17. Specifically, when the caregiver is in the presence of the patient, the caregiver mobile device 17 can be used to extract the identification code associated with a monitoring device 15 assigned to the patient. For instance, the mobile device 17 can automatically scan the barcode on a rounds tag worn by the patient or utilize NFC technology to communicate with a wheelchair sensor 15-1. The data associated with the event (i.e. the time, caregiver ID and patient ID associated with the visit) is then transmitted to central controller 19 and logged in database 21.

Third, as referenced above, a patient can request immediate caregiver attention by activating call button 119-1. The data associated with the call request (i.e. the time of the request and patient ID) is then transmitted to central controller 19 and logged in database 21. In this manner, system 11 is able to monitor the call request history for each patient as well as caregiver response times.

As principal feature of the present invention, the mobile device 17 utilized by each caregiver is specifically designed to directly receive important alerts and notifications relating to the group of patients under his/her care. In this manner, a patient in distress can be helped before engaging in activity which may result in significant injury.

For instance, if a patient has not been visited for a period of time that approaches the predefined patient rounds threshold, a rounds alert can be sent by patient monitoring center 13 to caregiver (via mobile device 17) by means of a visual, auditory and/or vibratory alarm. Similarly, if a patient is visited by a first caregiver, a second caregiver may receive a notification of the rounds visit, thereby enabling the second caregiver to provide services to other patients in need.

As another example, if wheelchair sensor 15-1 detects a low alert condition, color coded as yellow, an alarm with a unique auditory and/or vibratory cadence can be sent to a caregiver via mobile device 17. As such, the caregiver would be instantly notified (i.e. without even requiring removal of the mobile device from his/her pocket) that the patient, who was previously identified by name or another discernible descriptor during the setup process, may attempt to exit the chair and should be immediately assisted to prevent the risk of a fall. To assist in locating the patient, a buzzer (not shown) may be incorporated into device 15-1 to provide an auditory alarm in the immediate environment of the wheelchair.

Basic alert and notification settings for a particular mobile device can be easily modified using one or more touchscreen controls. Specifically, referring now to FIG. 6(d), there is shown a sample patient status screen display which is useful in understanding how a caregiver can modify certain alerts and notifications, the sample screen display being represented by reference numeral 531.

As can be seen, a dropdown box 533 has been activated which includes (i) an edit patient name button 535-1, an edit round time button 535-2, an edit red alarm delay button 535-3, an edit audio alarms button 535-4, an edit vibrate alarms button 535-5, a remove chair button 535-6 for removing a wheelchair sensor 15-1 from system 11, and an exit button 535-7 for closing dropdown box 533.

Referring now to sample screen display 537 shown in FIG. 6(e), activation of button 535-1 opens an edit patient name window 539 through which a caregiver can change the patient name associated with a particular monitoring device 15. Similarly, as provided in sample screen display 541 shown in FIG. 6(f), activation of button 535-2 opens an edit rounds timer window 543 through which a caregiver can modify the frequency with which rounds are to be conducted for associated patients. Further, as provided in sample screen display 545 shown in FIG. 6(g), activation of button 535-3 opens an edit red alarm display window 547 which enables a caregiver to incorporate a delay in transmitting a red alarm condition (e.g. for non-primary caregivers or to accommodate for expected alarms with a particular patient).

As noted above, the particular types of alarms which are programmed to run through a compute device 17 can be adjusted based on caregiver preferences. For example, as provided in sample screen display 549 shown in FIG. 6(h), activation of button 535-6 opens a check box control window 551 that enables the caregiver to specify whether an audio alarm should be generated upon the detection of certain conditions, such as a yellow alert condition, a red alert condition, a blue alert condition, and an off-line alert condition. Additionally, as provided in sample screen display 553 shown in FIG. 6(i), activation of button 535-7 opens a check box control window 555 that enables the caregiver to specify whether a vibratory alarm should be generated upon the detection of certain conditions, such as the aforementioned yellow, red, blue and off-line alert conditions.

In conclusion, it is clear that the software application installed on each mobile device 17 provides a highly intuitive means for a healthcare provider to track the status of multiple patients in real time. Through the use of auditory, vibratory and visually color-coded alerts, the caregiver can easily and effectively determine through its user interface the status of a plurality of patients and immediately administer attention to any patient who is potentially in need of assistance.

### Features and Advantages of the Present Invention

The system for monitoring a plurality of patients, as set forth in detail above, yields a number of notable advantages over traditional patient monitoring systems.

As a first advantage, system 11 is a comprehensive, all-in-one, web-based, patient monitoring solution that aggregates all patient data into a centralized database, the patient data including both information compiled from multiple monitoring devices 15 as well as observations rendered by various caregivers. This complete view of patient data allows for greater evaluation of the level of care provided with respect to institutional standards and, in turn, the minimization of potentially harmful conditions.

As a second advantage, system 11 is easily scalable, with any designated caregiver able to connect to system 11 by simply loading the software application for the platform onto his/her mobile device. In a similar fashion, patients can be added to system 11 through registration of one or more patient monitoring devices 15 (e.g. patient tag 15-2). In this manner, system 11 requires minimal data entry and is largely self-administering.

As a third advantage, system 11 utilizes a highly intuitive user interface through which a caregiver can track the status of multiple patients. Using a combination of auditory, vibratory and color-coded alerts, the caregiver can easily and effectively determine the status of a plurality of patients and immediately administer attention to any patient who is potentially in need of assistance.

As a fourth advantage, system 11 is able to monitor the real time posture, position and movement of a patient using patient monitoring device 15-1 or similar other constructs. In turn, system 11 is able to provide direct, real-time alerts of concerning patient movement to selected caregivers. As a result, system 11 effectively provides a proactive and anticipatory fall prevention solution, rather than simply notify of a fall condition after it has occurred.

The embodiment shown above is intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A system for monitoring of the status of a plurality of patients by a plurality of caregivers, a selection of the plurality of caregivers being assigned to each of the plurality of patients, the system comprising:
(a) a central controller for aggregating and evaluating data relating to the status of each of the plurality of patients;
(b) a plurality of patient monitoring devices, each of the plurality of patient monitoring devices being in close proximity to one of the plurality of patients, the plurality of patient monitoring devices being in electronic communication with the central controller; and
(c) a plurality of compute devices, at least one of the plurality of compute devices being in close proximity to a corresponding caregiver, the plurality of compute devices being in electronic communication with the central controller;
(d) wherein the central controller is adapted to receive data relating to the status of each of the plurality of patients from at least one of the plurality of patient monitoring devices and the plurality of compute devices, the central controller selectively transmitting alerts and notifications relating to the status of a patient to the corresponding selection of assigned caregivers for the patient via one or more designated compute devices.

2. The system of claim 1 wherein the central controller has direct access to a modifiable table that identifies the selection of the plurality of caregivers assigned to each of the plurality of patients, the at least one patient monitoring device assigned to each of the plurality of patients, and the at least one caregiver assigned to each of the plurality of compute devices.

3. The system of claim 2 wherein the table can be modified by each of the plurality of caregivers using one of the plurality of compute devices.

4. The system of claim 2 wherein the central controller evaluates the data relating to the status of each patient and identifies patient conditions which suggest an increased risk of falling.

5. The system of claim 4 wherein the central controller identifies patient conditions which suggest an increased risk of falling at different danger levels.

6. The system of claim 5 wherein an alert of the increased risk of falling is directly transmitted by the central controller to one or more of the plurality of compute devices in real time.

7. The system of claim 6 wherein the alert is represented by the one or more of the plurality of compute devices as at least one of a visual, an auditory and a vibratory alarm.

8. The system of claim 7 wherein the alert represented on the one or more of the plurality of compute devices identifies the danger level of falling.

9. The system of claim 8 wherein the visual alarm represents the danger level of falling using predefined color codes.

10. The system of claim 2 wherein the central controller has access to historical data relating to the status of each of the plurality of patients.

11. The system of claim 1 wherein a first of the plurality of patient monitoring devices comprises:
(a) an outer housing shaped to define an interior cavity and at least one slot;
(b) electronics disposed within the interior cavity for controlling the principal operations of the first of the plurality of patient monitoring devices; and
(c) at least one motion sensor disposed within the interior cavity in alignment with the at least one slot, the at least one motion sensor being in electrical communication with the electronics;
(d) wherein the at least one motion sensor is adapted to monitor the position of a patient and send a corresponding signal to the electronics.

12. The system of claim 11 wherein the first of the plurality of patient monitoring devices is adapted to be releasably secured to a wheelchair having a seat.

13. The system of claim 12 wherein the first of the plurality of patient monitoring devices detects initiation of patient egress from the wheelchair to which it is secured.

14. The system of claim 13 wherein the first of the plurality of patient monitoring devices detects multiple stages of initiation of patient egress from the wheelchair to which it is secured based on the region of the seat of the wheelchair on which the patient is sitting.

15. The system of claim 14 wherein the electronics comprises a transmitter for wirelessly sending patient monitoring data to the central controller.

16. The system of claim 15 further comprising at least one externally accessible control button for manually regulating certain operations of the first of the plurality of patient monitoring devices, the at least one control button being mounted in the outer housing in electromechanical communication with the electronics.
